(19) United States
(12) Patent Application Publication
(10) Pub. No.: US 2017/0000993 A1
(43) Pub. Date: Jan. 5, 2017

(54) FILTER-AND-MEDICATION TYPE SHOWER ARM

(71) Applicant: Xiamen Runner Industrial Corporation, Xiamen (CN)

(72) Inventors: Wen-kun Lin, Xiamen (CN); Yuan-shan Liang, Xiamen (CN); Jian-ke Huang, Xiamen (CN)

(21) Appl. No.: 15/189,000

(22) Filed: Jun. 22, 2016

(30) Foreign Application Priority Data
Jun. 30, 2015 (CN) 201520467975.5

(51) Int. Cl. A61M 35/00; B01D 35/02; C02F 1/68; E03C 1/04; C02F 1/00

(52) U.S. Cl. CPC A61M 35/00; E03C 1/0408; C02F 1/001; C02F 1/68; B01D 35/02; A61M 2205/75; C02F 2103/026

(57) ABSTRACT

A filter-and-medication type shower arm includes a filter portion and a medication portion. The filter portion includes: a shower arm water input port, disposed at end of the filter portion; and a filter core, disposed in the filter portion, with its one end connected to the shower arm water input port. The medication portion is connected to the filter portion, so that they are in communication with each other, including: a shower arm water output port, disposed at end of the medication portion, and is provided with a shower head connector; a chamber, connected to other end of the filter core; and a medicine storage box, disposed slidably in the chamber of the medication portion, and containing a medicine bag inside, and it includes: a plurality of micro through holes, and a water outlet port.

### BACKGROUND OF THE INVENTION

Field of the Invention

The present invention relates to a shower arm, and in particular to a filter-and-medication type shower arm.

The Prior Arts

In recent years, people have high expectations and demands for the shower equipment. To meet this demand, manufacturers have put filter core and medicine bag in the shower arm. As such, in taking a shower, the shower arm may provide filtered clean water having medicine treatment effect. However, in case the water quality is poor, a filter core tends to get blocked quickly. If the shower arm is blocked and can not be replaced, then the shower arm is rendered ineffective. Therefore, for a filter type shower arm, the filter core has to be replaced easily. Yet, for a shower arm, a too complicated structure tends to adversely affect its water output. In addition, in taking a shower, the medicine bag in the shower arm is only immersed in the flowing water for a short period of time, so that the medicine immersed out is quite limited, the user may not get sufficient effect of the medicine. Therefore, it is important that, for a shower arm, the filter core has to be designed to be replaced easily, while the user may have sufficient effect of the medicine.

Therefore, presently, the design of shower arm is not quite satisfactory, and it leaves much room for improvement.

### SUMMARY OF THE INVENTION

In view of the problems and drawbacks of the prior art, the present invention provides a filter-and-medication type shower arm, to overcome the shortcomings of the prior art.

The major objective of the present invention is to provide a filter-and-medication type shower arm, which contains a filer core and a medicine bag. Its structure design is simple, thus its filter core can be replaced easily. Further, the wall of its medicine storage box is designed to have through holes, to produce bubbles for the water flowing through, and to mix them in the water to flow in random directions, to enhance the immersing out capability of the medicine in the medicine bag, so that the user may get sufficient effect of the medicine.

The present invention provides a filter-and-medication type shower arm, comprising: a filter portion and a medication portion.

The filter portion includes: a shower arm water input port, disposed at end of the filter portion; and a filter core, disposed in the filter portion, with its one end connected to the shower arm water input port. The medication portion is connected to the filter portion, so that they are in communication with each other, including: a shower arm water output port, disposed at end of the medication portion, and is provided with a shower head connector; a chamber, connected to other end of the filter core; and a medicine storage box, disposed slidably in the chamber of the medication portion, and containing a medicine bag inside, and it includes: a plurality of micro through holes, disposed densely on its outer wall, for water to pass through; and a water outlet port, disposed on a wall of the medicine storage box facing the shower arm water output port.

In an aspect of the present invention, a large number of micro through holes are distributed evenly on an outer wall of the medicine storage box, and is used for cutting water, so that water flows in the medicine storage box in random directions.

In another aspect of the present invention, gap exists between the medicine bag and an inner wall of the medicine storage box.

In yet another aspect of the present invention, the medicine bag in the medicine storage box is composed of a plurality of small medicine bags.

In a further aspect of the present invention, the medicine bag contains herb medicine or essence.

In another aspect of the present invention, the filter-and-medication type shower arm further comprises: a magnetic ring, disposed at one end of the filter core connecting the chamber.

In yet another aspect of the present invention, the filter-and-medication type shower arm further comprises: a core fetching rod made of magnetic material, used to fetch the filter core from the filter portion, in cooperation with the magnetic ring.

In a further aspect of the present invention, an outer wall of the medicine storage box is composed of multi layers.

In another aspect of the present invention, the filter-and-medication type shower arm further comprises: a spiral, through which the filter portion is connected to the medication portion, such that the filter core is inserted and fixed in the filter portion in an extractable way.

In the present invention, a large number of micro through holes are disposed on the outer wall of the medicine storage box to cut water flow. The outer wall of the medicine storage box is composed of multi-layers, such that water flowing through into the medicine storage box is made to flow in random directions. In the medicine bag of the medicine storage box is provided with herb medicine or essence, while gap exists between the medicine bag and the medicine storage box. The structure of the medicine storage box outer wall, is similar to that of the bubbler of the faucet. When water flows through the multi layer outer wall, it will be cut by the micro through holes multiple times, so that the air in the medicine storage box will be merged into water flow in a form of bubble, to produce bubbled water having large number of bubbles. As such, when the bubbled water enters into the medicine storage box, not only the herb medicine contained therein can be immersed and dissolved effectively into water, but the essence in the medicine bag can also be volatilized into the bubble, to produce large number of bubbles having fragrance of essence. Therefore, when the bubbled water having herb medicine and essence flow out from the shower head, the fragrance of essence is dissipated quickly into the air of the bath room. Since the essence carrying effect produced by water bubbling is better than that as done by water immersing. As such, in taking a shower, the user may enjoy the fragrance of the essence, while getting sufficient effect of the medicine.

In the medicine storage box, the medicine bag is formed by a plurality of small bags, while the micro through holes are distributed on the outer wall of the medicine storage box. The micro through holes are capable of making the water to flow into random directions, so that the medicine bag is flushed by water flows of various directions. Since the medicine bag is made of small bags of light weight, thus in the flush of random direction water flows, they can be turned violently to raise the effect of immersing and extracting out the medicine.

In the present invention, filter core is provided, such that the water input to the shower arm will first be filtered by the filter core, and only after that process, the water then flows into the medicine storage box. As such, particles and impurities are filtered out by the filer core, and will not block the micro through holes of the medicine storage box, to avoid the shortcomings of the ordinary bubbler being liable to be blocked.

In the present invention, a magnetic ring is provided at one end of the filter core, that is capable of absorbing metal particles in the water, and magnetizing the water flow. After magnetization, the water solution capability is enhanced, hereby raising the immersing out effect of the medicine in the medicine bag.

In the present invention, the spiral is provided to connect the filter portion and the medication portion, so that the two portions are in communication with each other. The filter core is placed at the position where the filter portion connects the medication portion, such that the filter core can be replaced easily. When replacing the filter core, all it has to do is to unscrew the medication portion, then the filter core can be reached and replaced.

In the present invention, a core fetching rod made of magnetic material is further provided, so that when it is required to replace the filter core, the core fetching rod can be used to absorb the magnetic ring on the filter core, then pull the core fetching rod to pull out the filter. In this approach, the structure outside the filter core can be simplified, and there is no need to place a handle on the filter core. As such, it can reduce the production cost of the filter core, while making the structure of shower arm to be more compact, and be more aesthetically pleasing.

The medicine storage box is slidable in the chamber of the medication portion, such that the medicine storage box can be replaced easily. In the replacing process, all it has to do is to unscrew the medication portion, then pull out the medicine storage box from the place near the spiral.

Further scope of the applicability of the present invention will become apparent from the detailed descriptions given hereinafter. However, it should be understood that the detailed descriptions and specific examples, while indicating preferred embodiments of the present invention, are given by way of illustration only, since various changes and modifications within the spirit and scope of the present invention will become apparent to those skilled in the art from the detailed descriptions.

### BRIEF DESCRIPTION OF THE DRAWINGS

The related drawings in connection with the detailed descriptions of the present invention to be made later are described briefly as follows, in which:

FIG. 1 is a schematic diagram of a filter-and-medication type shower arm according to an embodiment of the present invention.

### DETAILED DESCRIPTION OF THE PREFERRED EMBODIMENT

The purpose, construction, features, functions and advantages of the present invention can be appreciated and understood more thoroughly through the following detailed descriptions with reference to the attached drawings.

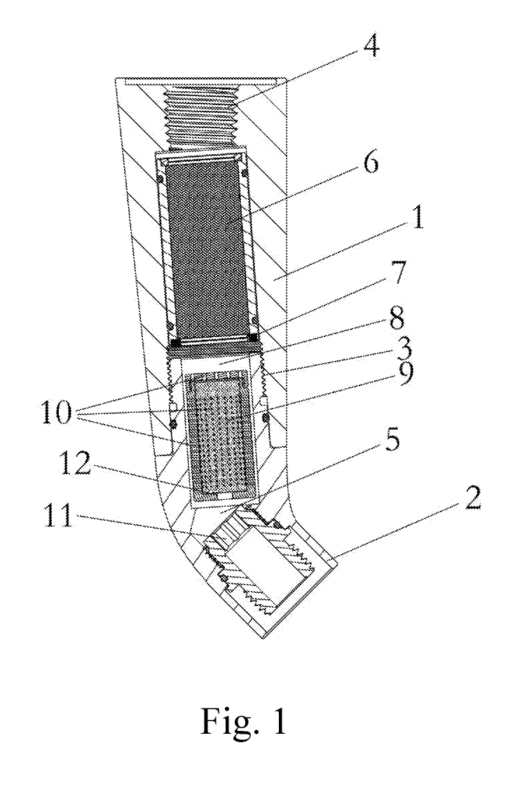

Refer to FIG. 1 for a schematic diagram of a filter-and-medication type shower arm according to an embodiment of the present invention. As shown in FIG. 1, the present invention provides a filter-and-medication type shower arm, comprising: a filter portion 1 and a medication portion 2.

The filter portion 1 includes: a shower arm water input port 4, disposed at end of the filter portion 1; and a filter core 6, disposed in the filter portion 1, with its one end connected to the shower arm water input port 4. The medication portion 2 is connected to the filter portion 1, so that they are in communication with each other, including: a shower arm water output port 5, disposed at end of the medication portion 2, and is provided with a shower head connector 11; a chamber 8, connected to other end of the filter core 6; and a medicine storage box 9, disposed to be slidable in the chamber 8 of the medication portion 2, and containing a medicine bag inside, and it includes: a plurality of micro through holes 10, disposed densely on its outer wall, for water to pass through; and a water outlet port 12, disposed on a wall of the medicine storage box 9 facing the shower arm water output port 5.

In an embodiment of the present invention, a large number of micro through holes 10 are distributed evenly on an outer wall of the medicine storage box 9, and is used for cutting water, so that water flows in the medicine storage box 9 in a random direction.

In an embodiment of the present invention, gap exists between the medicine bag and an inner wall of the medicine storage box 9.

In an embodiment of the present invention, the medicine bag in the medicine storage box 9 is composed of a plurality of small medicine bags.

In an embodiment of the present invention, the medicine bag contains herb medicine or essence.

In an embodiment of the present invention, the filter-and-medication type shower arm further comprises: a magnetic ring 7, disposed at one end of the filter core 6 connecting the chamber 8.

In an embodiment of the present invention, the filter-and-medication type shower arm further comprises: a core fetching rod made of magnetic material, used to fetch the filter core 6 from the filter portion 1, in cooperation with the magnetic ring 7.

In an embodiment of the present invention, an outer wall of the medicine storage box 9 is composed of multi layers.

In an embodiment of the present invention, the filter-and-medication type shower arm further comprises: a spiral 3, through which the filter portion 1 is connected to the medication portion 2, such that the filter core 6 is inserted and fixed in the filter portion 1 in an extractable way.

### Embodiment 1

In application, a water pipe is connected to the shower arm water input port 4. After water enters into the shower arm, the filter core 6 filters out the particles and impurities from the input water, then the magnetic ring 7 magnetizes the water and absorbs the rust from the water. When water reaches the chamber 8 of the medication portion 2, the water pressure pushes the medicine storage box 9 to move, to press against the shower arm water output port 5. At this time, in the chamber 8, water is cut multiple times, and then it flows through the micro through holes on the outer wall into the medicine storage box 9. In this process, it produces the same effect as the faucet bubbler, so air is mixed into water in a form of bubbles, to generate bubbled water containing large number of bubbles. Meanwhile, water is cut by the micro through holes to flow in random directions. The bubbled water keeps turning and rowing in the medicine storage box 9, and it brings the medicine bag into turning and rowing, to cause the medicine contained therein to dissolve effectively into water. Further, the fragrance of the essence in the medicine bag is volatilized into the bubbles, to produce large number of bubbles having fragrance. As such, bubbled water having medicine and fragrance flows to the shower arm water output port 5 through the water outlet port 12.

### Embodiment 2

In application, when it is not required to generate bubbled water for the medicine storage box 9, then in installing a shower head, a shower head having greater water resistance, such as high water pressure shower head can be selected, to be connected to the shower head connector 11. Therefore, when taking a shower, the water output amount of the shower arm is reduced, due to greater water resistance of the shower head. As such, the medicine storage box 9 is full of water, bubbles are not liable to produce.

### Embodiment 3

When the filter core 6 has been used for a period of time, the holes on it tend to be blocked to affect its water output. In replacing the filter core 6, all it has to do is to unscrew the medication portion 2 from the filter portion 1, next utilize the core fetching rod to touch and absorb the magnetic ring 7, then pull out the core fetching rod to pull out the filter core 6 along, and finally put in the new filter core 6 to complete replacement.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A filter-and-medication type shower arm, comprising:
a filter portion, including:
a shower arm water input port, disposed at end of the filter portion; and
a filter core, disposed in the filter portion, with its one end connected to the shower arm water input port; and
a medication portion, connected to the filter portion, so that they are in communication with each other, including:
a shower arm water output port, disposed at an end of medication portion, and provided with a shower head connector;
a chamber, connected to other end of the filter core; and
a medicine storage box, disposed slidably in the chamber of the medication portion, and containing a medicine bag inside, including:
a plurality of micro through holes, disposed densely on its outer wall, for water to pass through; and
a water outlet port, disposed on a wall of the medicine storage box facing the shower arm water output port.

2. The filter-and-medication type shower arm as claimed in claim 1, wherein a large number of micro through holes are distributed evenly on an outer wall of the medicine storage box, and is used for cutting water, so that water flows in the medicine storage box in random directions.

3. The filter-and-medication type shower arm as claimed in claim 1, wherein gap exists between the medicine bag and an inner wall of the medicine storage box.

4. The filter-and-medication type shower arm as claimed in claim 1, wherein the medicine bag in the medicine storage box is composed of a plurality of small medicine bags.

5. The filter-and-medication type shower arm as claimed in claim 1, wherein the medicine bag contains herb medicine or essence.

6. The filter-and-medication type shower arm as claimed in claim 1, further comprising: a magnetic ring, disposed at one end of the filter core connecting the chamber.

7. The filter-and-medication type shower arm as claimed in claim 6, further comprising: a core fetching rod, made of magnetic material, used to fetch the filter core from the filter portion, in cooperation with the magnetic ring.

8. The filter-and-medication type shower arm as claimed in claim 1, wherein an outer wall of the medicine storage box is composed of multi layers.

9. The filter-and-medication type shower arm as claimed in claim 1, further comprising a spiral, through which the filter portion is connected to the medication portion, such that the filter core is inserted and fixed in the filter portion in an extractable way.
